(19) United States
(12) Patent Application Publication
(10) Pub. No.: US 2008/0089817 A1
(43) Pub. Date: Apr. 17, 2008

(54) Anti-bacterial bomb for area broadcast

(76) Inventor: Robert James Cacciabaudo, Titusville, NJ (US)

Correspondence Address: CHRISTOPHER D. HARRINGTON, SUITE 350, 5300 CORPORATE GROVE DR, SE, GRAND RAPIDS, MI, 49512, US

(21) Appl. No.: 11/581,254

(22) Filed: Oct. 16, 2006

(51) Int. Cl. A61L 2/22

(52) U.S. Cl. 422/292

(57) ABSTRACT

A novel bomb spray for use in disinfecting a room is disclosed where the bomb spray includes a reservoir and a spray nozzle where the contents of the reservoir include a disinfectant chemistry that is dischargeable through the spray nozzle. The spray nozzle is optimized to discharge all of the contents of the reservoir and to project the disinfectant chemistry simultaneously in a vertical and radial pattern in order to broadcast the disinfectant spray on all of the surfaces in the room. The disinfectant chemistry is selected from formulations that contain some amount of a quaternary ammonium compound for imparting bactericidal properties. In addition, the disinfectant chemistry includes a volatile matrix that promotes rapid drying of the disinfectant after it has been dispersed into the room.

### CROSS REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

The present invention relates to a disinfectant bomb for use in providing immediate broadcast of a chemical disinfectant. More particularly, the present invention relates to a disinfectant bomb for use in confined and semi-confined areas for the immediate disinfection of surfaces.

In today's clinical environment the high demand for working space is at an all high. In particular, examining rooms, surgical rooms and patient rooms are very expensive and most hospitals and clinics seek to utilize these spaces to the maximum possible potential. Given the high turnover rates therefore, there is a demand on staff to prepare each of these rooms and restore them to the required conditions before the next patient is handled. Depending on the institution and the particular parameters that each is obliged to use, this can mean that working surfaces such as table tops, trays, examining tables, gurneys or beds, are typically cleaned to a point they are considered disinfected. This can be through the use of chemicals sprays or towelettes that have been saturated with disinfectant chemistry, or other methods that help restore the area to the required levels. This effort is labor intensive and can still result in some surfaces in the area of concern to remain untreated given the fact that it is not possible for the staff person to visibly see what surfaces have been cleaned as required and which ones have not.

In may in fact be desirable for the hospital or clinic to cause more surface area than is typically set forth as the requirement for cleaning. This often time will include walls and floors and ceilings which may be cleaned and disinfected on a different frequency than the working surfaces in the particular room, notwithstanding the fact that wholesale disinfection of the room would be preferred. The investment in labor and time militates against the best possible conditions in such cases in favor of conditions that may be considered optimal, at best.

The usual examining, treatment or patient room does have some boundaries. In most cases the room is bounded by walls, sometime solid and sometime they may be moveable partitions. The room is bounded at the top by a ceiling which usually is a suspended ceiling containing acoustic tiles. The floors are usually tile floors which are resistant to periodic mopping and cleaning. The entry into such rooms may be through a framed door and doorway which also presents with smooth and durable surfaces.

Alternate forms of such rooms may have draped partitions to segregate one treatment or patient area from the other, however, the overall area is typically confined even if the individual rooms are not.

Chemical bombs of various types are known in the prior art. These are mainly directed for use against insects of various kinds, for instance, in U.S. Pat. No. 5,246,675 (Castronovo) discloses a bomb type canister for use in an aperture in a ceiling. The aperture opens up into the attic area and allows the user to activate the bomb from the protection below the ceiling. In U.S. Pat. No. 5,503,303 (LaWare, et al) the bomb is comprised of an aerosol can with an overcap that provides the desired spray for an insecticide. Similarly, in U.S. Pat. No. 4,568,002 (Weinstein, et al) an aerosol canister with a special nozzle for the distribution of an insecticide is shown. The overcap is again the primary consideration in U.S. Pat. No. 4,426,025 (Knickerbocker) where the overcap provides protection for the nozzle during shipment, thereby preventing accidental activation.

The usage of select quaternary ammonium compounds in a matrix of supporting chemistry has been shown to be an effective disinfectant and these are commonly found in various preparations for home and industrial uses. Many of these preparations are packaged in standard aerosol spray containers for direct spray cleaning of various surfaces typically suited for sanitizing a bathroom area or work surfaces where disinfection is needed. An example of such a composition that may be used is disclosed in U.S. Pat. No. 4,125,628 (Goldhaft, et al) where a specific groups of formulations employing quaternary ammonium compounds are explained. Similar chemistry is also shown in U.S. Pat. No. 4,540,505 (Frazier) where a slightly different formulation is used in a matrix that includes quaternary ammonium compounds.

In U.S. Design Patent No. D480,629 (Cabrera) an ornamental design for a “fogger canister” is shown with a label suggesting it is to be used with a disinfectant. The type of disinfectant is not disclosed in Cabrera although a fogger is a distinctly different type of aerosol than one that is used as a bomb. The difference arises from the type of action that is generated by the spray head. In a fogger, the chemicals are atomized in extremely small sizes such that the chemistry remains suspended in the air for a long period of time. The longer hang time for the chemistry allows a greater degree of contact between anything that might be in the air and the chemistry that is being distributed. In the case of an insecticide, which is commonly supplied as a “fog” type product, the increased contact time increases the effectiveness of the kill rate that is obtained especially for flying insects. With respect to a disinfectant, the greater degree of contact time that is achieved using a fog type spray system may be beneficial for taking down bacterial counts in a specific atmosphere, but this method is counter-productive when the objective is to distribute the disinfectant onto surfaces within a confined area for a quick sanitizing treatment that will allow the room to be returned to service as soon as possible with a minimum of effort.

The longstanding problems associated with the ability to achieve a thorough disinfection of a confined area, such as an examining room, have continued to plague the medical community, veterinary clinics and animal hospitals, nursing homes, and other similar institutions. The benefits and attributes of the present invention, as will be discussed in detail below, provide a solution to these problems in a cost effective and very efficient manner.

### SUMMARY OF THE INVENTION

A novel bomb spray for the disinfection of surfaces within a room, comprises a bomb canister with a reservoir for holding a disinfectant chemistry, and a spray nozzle for generating a spray, where the spray nozzle provides a 360 degree broadcast for comprehensive distribution of the disinfectant chemistry. The broadcast of the disinfectant allows substantial surface area within the room to be contacted by effective concentrations of the disinfectant chemistry, thereby knocking down the bacterial counts on said surfaces. The bomb spray of the present invention discharges its contents in a matter of seconds, with the spray characteristics optimized for broadcast of the chemistry within the confined space and for quick contact with the targeted surfaces.

The disinfectant chemistry of the bomb spray of the present invention is also optimized for fast drying characteristics, providing dry surfaces within a short time following the activation of the bomb spray thereby allowing the room to be returned to use in a quick fashion.

### BRIEF DESCRIPTION OF THE DRAWINGS

FIG. 1 is an isometric view of a bomb spray of the present invention showing the reservoir and the spray nozzle components.

FIG. 2 is a diagrammatic representation of a general formulation for use in the $ bomb spray of the present invention.

FIG. 3 is a cross sectional view of the spray nozzle of the bomb spray as depicted in FIG. 1, taken along Section Lines 3-3.

FIG. 4 is an isometric view of an examining room with a bomb spray of the present invention placed and ready for activation.

FIG. 5 is the examining room of FIG. 4 with the bomb spray activated and the broadcast of the spray shown in phantom lines.

### DETAILED DESCRIPTION OF THE PREFERRED EMBODIMENTS

A bomb spray in accordance with the present invention is shown in the drawings and is discussed in detail within this specification. The bomb spray is based upon the use of a disinfectant that is compatible for use in areas that will be occupied at times by humans and/or animals. As will be seen, the disinfectant chemistry that is suitable for selection with the present invention is limited both for its effectiveness in achieving a sanitary condition, but also for its ability to work in a specified space and in a time frame that meets the objectives of the user.

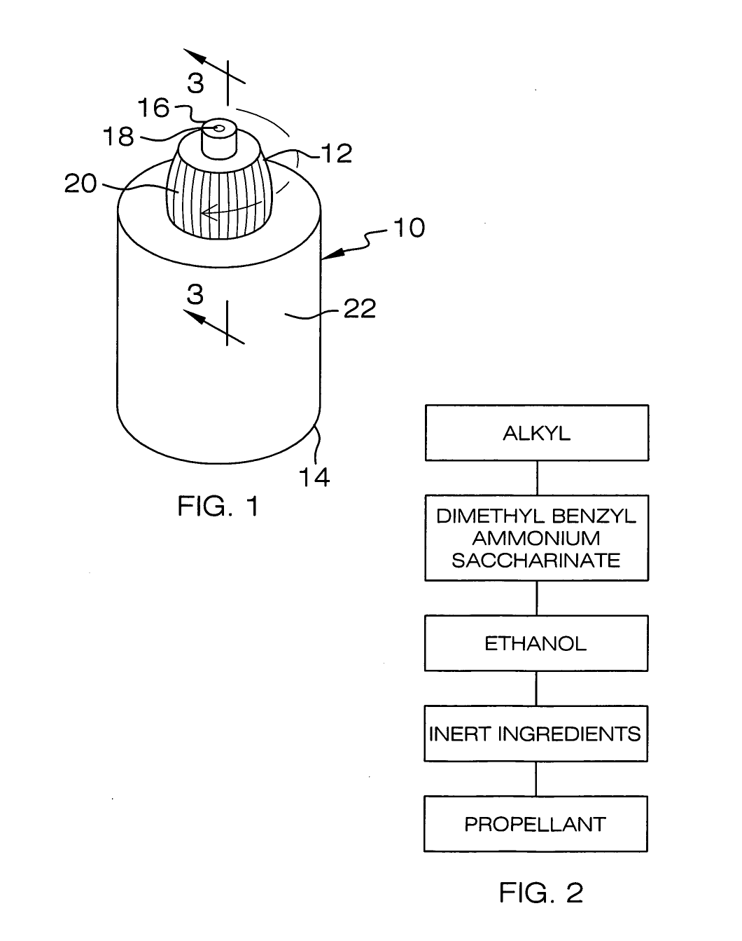

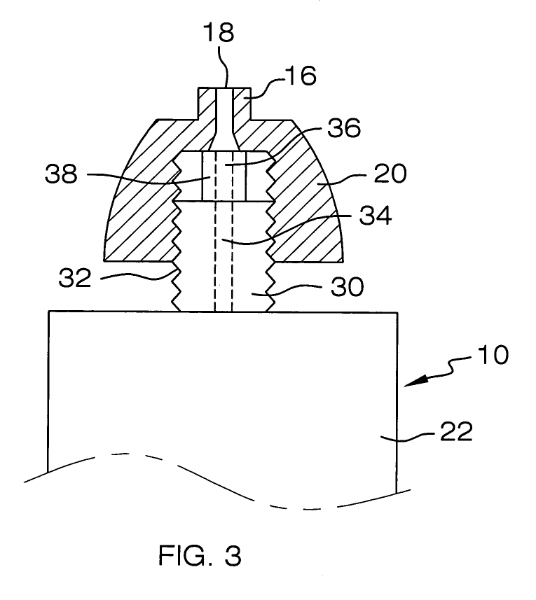

Turning now to FIG. 1, a bomb spray 10 in accordance with the present invention is shown with the spray nozzle 12, the bomb spray bottom 14, the nozzle body 16, the spray port 18, the control collar 20 and the reservoir 22. More of the bomb spray 10 structure is visible in FIG. 3 with the reservoir fitting 30, the fitting threads 32, the discharge port 34, the intermediate port 36 and the intermediate port body 38.

As may now be appreciated, the bomb spray functions like a conventional aerosol spray up to the point of the spray function. For this reason, some of the structure relating to conventional aerosol sprays is not shown although it is understood that it does contribute to the functionality of the present invention, albeit in a known manner. The disinfectant chemistry is contained within the reservoir is under pressure and which includes a propellant for this purpose. The reservoir is in fluid communication with the discharge port and is prevented from existing the spray nozzle by means of the intermediate port that is located eccentrically within the control collar, the combination of the control collar and the intermediate port being rotateable into the position shown in FIG. 3 to allow flow to occur between the chemicals in the reservoir and the spray nozzle. The control collar controls the operation of the bomb spray, therefore, operating between a stored condition where the spray is prevented for being discharged to an activated condition where the disinfectant chemistry is discharged through the spray nozzle. The present invention is preferably contemplated as a single use type of container which means that the spray nozzle assembly does not need to be sensitive to issues of multiple uses or long shelf life. It should be understood that the reservoir is typically comprised of an aluminum or steel canister, the selection being more a matter of economics. As will be discussed below, the size of the canister may vary depending on the intended application.

Once the intermediate port is in position to allow the contents of the bomb spray to be discharged; the flow exits the spray port. The objective of the spray port in the present invention is to provide a quick discharge of the contents while broadcasting the resulting spray that evolves from the spray port into a 360 degree pattern. The chemical spray will be ejected from the bomb of the present invention in a manner similar to other bombs where the subject chemical is projected above the bomb spray both vertically in height and radially. If taken in isolation, if the bomb spray were activated in a confined area, the spray pattern would ideally fill the volume of the area. In contrast to a fogger type of spray, however, that is caused to intentionally linger because of the smaller drop sizes that are achieved the bomb spray of the present invention is meant to project the chemistry into as much of the confined areas as possible, contacting as many surfaces as is possible, and then succumbing to gravity and settling out. The process is preferentially intended to be brief allowing for the time necessary to discharge the contents of the bomb spray into the confined area and then settling out soon thereafter.

The spray nozzle and the conditions of pressure within the reservoir can be adjusted to maximize the objectives of the present invention and this relationship can be managed by one skilled in the art of manufacturing bomb sprays. Unlike other bomb sprays though, the effectiveness of this concept is not dependent upon contact while the chemistry is airborne since the usual bomb spray is directed towards the knock down of insects. That is not to say that there aren't benefits while the disinfectant chemistry of the present invention is airborne, it presumably will have an effect on airborne bacteria and pathogens, however it is the effect that the disinfectant chemistry has when it contacts the surface within the confined space, such as an examining room.

For the purposes of illustrating the invention, an examining room in a hospital or clinic setting will be used although it is understood that this is not the exclusive application for the bomb spray which may be used in a number of situations where the goal is to provide a quick sanitizing of a room or similar space. The examining room, as would other scenarios, has a number of surfaces that can harbor bacteria and pathogens. The prevalence of bacteria and pathogens in an environment like an examining room is understood since the room is used for the examination and treatment of numbers of patients within its confines. Thus it is known that bacteria and pathogens can be literally found on all surfaces within a given space like an examining room, although there is a difference between the surfaces. For instance, there are working surfaces that are primary surfaces to contend with. These are surfaces associated with table tops, beds or gurneys, countertops, and the like. They are usually the places where the staffer or patient is found and where procedures that involve the evolution of bacteria and/or 2Q pathogens can take place. Consider a table surface that was the location for the suturing of a wound, which becomes contaminated after the procedure notwithstanding the removal of drapes, tools, and trays. The residual occupation of bacteria and pathogens in such settings is of major concern to hospitals and clinics for the reason that the transfer of any bacteria or pathogens to subsequent patients can pose a real problem from a medial and a liability standpoint.

These working or primary surfaces are typically horizontal with respect to the examining room and are receptive to the deposition of disinfectant chemistry from a bomb spray. Thus the contact time associated with the disinfectant and the working surface is maintained and easily surpasses that which is needed to achieve an adequate knock down of bacteria and pathogens on those surfaces.

There are other surfaces of concern within the examining room. These may be termed secondary surfaces and include the walls, the floor and the ceiling of the examining room. In the usual case, the secondary surfaces are not considered when the examining room is being prepped for a patient. As alluded to above, the working surface are usually prepared by the removal of drapes, trays, equipment, spent products such as gauze or tape, linens, and the like. The working surfaces are also usually given a wipe with a disinfectant product however depending on the attention to detail that is given this may be very ineffective in actually sanitizing all of the working surfaces. When it comes to the secondary surfaces the situation is even worse. There is no ability to completely prepare all of the surfaces within an examining room between use, and no attempt is made. Periodically the rooms are mopped, and less periodically the walls may be scrubbed. This does not mean that the bacteria and pathogens are not present on secondary surfaces; they are and sometimes in substantial amounts and types to raise real concerns about the foregoing method for preparing the examining room.

It is a function of the present invention to broadcast a disinfectant spray within the confines of a space like an examining room and to have a sanitizing effect on both the working (primary) surfaces and the secondary surfaces. The usage of the bomb spray in this regard is a method that is not an immediately intuitive approach since in the past the usage of fogs has been assumed to be the preferred route for functions relating to the disinfection of surfaces. The fog approach certainly does have merit in terms of the knock down capabilities within the air space of a confined area, however it persists in the airspace and it increases the turnaround time for the re-use of the treated room. By focusing on the primary and secondary surfaces within the room that need to be sanitized, the usage of the bomb spray provides a surprising and unexpected method for this task, although it is not just the bomb spray that provides the solution to the longstanding problems associated with the preparation of examining rooms and the like, the selection of the type of chemistry to be used impacts the effectiveness and objectives as well.

The preferred chemistry for use n the bomb spray of the present invention relies on a category of disinfectants associated with quaternary ammonium compounds. The chemical group of benzalkonium chlorides (alkyl dimethyl benzyl ammonium chloride) is comprised of a number of related compounds that are all classed within the quaternary ammonium group. Benzalkonium chloride is actually a mixture of alkylbenzyl dimethyl ammonium chlorides of various alkyl chain lengths. The greatest bactericidal activity is associated with the C-12 C-14 derivatives. Benzalkonium chlorides have shown to be effective against bacteria, and some viruses and fungi and protozoa. In addition, these compounds are generally considered to be safe for humans and have even been included in formulations intended for ingestion. The combination of efficacious and safe effects make for an effective selection of the benzalkonium chlorides for use in the bomb spray. One particular formulation that is known to the applicant to be effective is described in Table 1:

TABLE 1
Composition of Disinfectant Chemistry

| | |
|---|---|
| Alkyl dimethylbenzylammonium | 1% |
| saccharinate | |
| Alkyl groups: | |
| CH | 50% |
| C-12 | 40% |
| C-16 | 10% |
| Ethanol | 75% |
| Inert | 24–25% |

As can be appreciated, the volume of ethanol contributes to the rate at which the disinfectant evaporates, leaving behind the active component of the disinfectant chemistry which will continue to have a beneficial effect on any of the surfaces it contacts. Not included in the formulation is the propellant that is used for the bomb spray; however this is inert and does not form a part of the disinfectant chemistry. The propellant needs to be effective to discharge substantially all of the contents of the reservoir and this is a matter of selection for one skilled in the art of bomb spray manufacturing. The size of the bomb spray, that is the amount of disinfectant chemistry that is to be used, will vary with the size of the target room that is to be treated. These variations in the concept are merely engineering choices and may be determined on a case-by-case basis, or preferably standard sizes will be established for rooms approximating the average sized examining room.

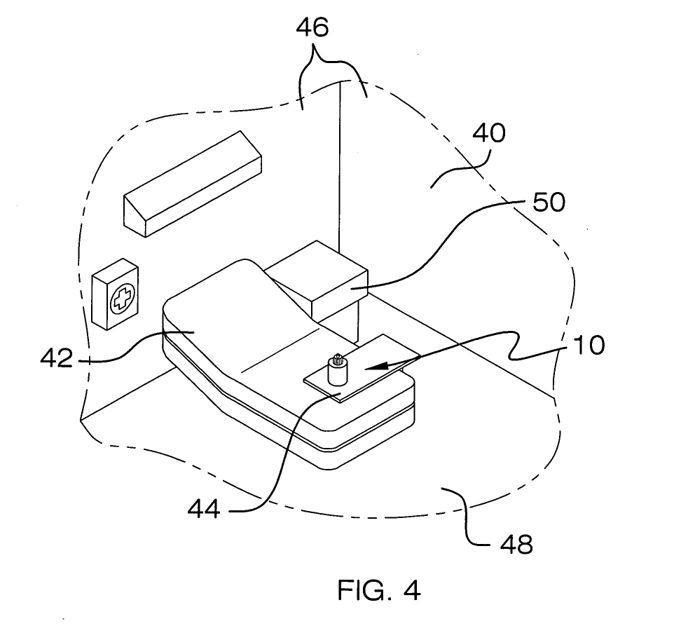

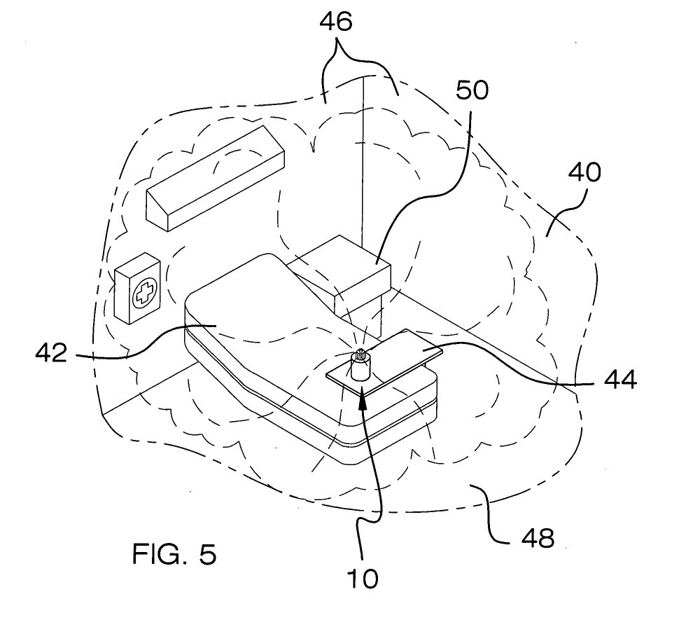

Turning now to FIGS. 4 and 5, an illustration of the use of the bomb spray of the present invention is shown in a typical examining room scenario. In FIG. 4, the bomb spray 10 is shown in the examining room 40 before activation. In the examining room 40 is the bed 42, the table 44, walls 46, floor 48 and cabinet 50 as would be the case for virtually any room of this type. The primary surfaces would be represented by the bed, the top of the table and the top of the cabinet. The secondary surfaces would be represented by the floor, the walls and the ceiling (not shown). The bomb spray is placed appropriately for use, in a location that is substantially near the center of the room and at a height calculated to allow the spray that is ejected to be able to fall onto or contact the primary and secondary surfaces in the room.

In FIG. 5 the bomb spray has been activated and a representation of the chemical dispersion is shown. As can be appreciated, the distribution is horizontally radial while the ejection of the spray from the spray nozzle will vertically distribute spray at the same time. The effect is to immediately distribute disinfectant chemicals throughout the room and as discussed above, the chemistry will very quickly settle down in direct contact with both primary and secondary surfaces. The fact that the primary surfaces will at times receive more disinfectant spray is a consequence of the horizontal orientation of many of those surfaces and gravity; however this is also the case when bacteria or pathogens are ejected from a source such as will happen when staff works with a patient at times. The same is true in the case of the floor which has been termed a secondary surface; however that terminology is only employed because of the priority given to the surface, which is lowered for the floor owing to the impracticality of scrubbing the floor after each use of the examining room. Bacteria and pathogens are projected from open wounds, sneezing, coughing, spit, and the like, and will have a distribution profile that is similar to that obtained by the bomb spray of the present invention.

The advantages of the present concept can be seen with respect to the ability to have a thorough sanitizing of the subject room. Secondary surfaces are normally not considered when preparing the examining room for the next use. With the advent of the present invention the secondary surfaces receive the benefit of a disinfectant treatment and in the case of the primary surfaces, the distribution of the disinfectant chemistry ensures a more thorough coverage than would otherwise be achieved by hand cleansing of the surfaces. The method employing the bomb spray of the present invention is important since it provides a quick solution to increasing the sanitary conditions in a facility such as a hospital or a clinic. In addition it is not labor intensive at all, it merely requires the placement of the bomb spray in the central portion of the room, activating the spray to cause it to discharge, and then waiting until the disinfectant chemistry has completed the distribution, settling and drying before allowing the room to be re-used. It is anticipated that the process can be reduced to a matter of seconds for an 8′ by 8′ examining room. A room like this would contain approximately 512 cubic feet of airspace and by experience a bomb spray of less than 8 ounces of disinfectant can be evolved to adequately fill this volume. In most cases the discharge would last for less than two or three minutes and the time for drying is about the same. In essence, by the time the staff is aware that the room is ready for use, the disinfection process has been completed. The bomb spray may be offered as a single-trip type of container/product where it is discarded once it has been used. In the alternate, the bomb spray may be collected and recycled by a manufacturer merely by removing the spray nozzle (refurbishing if necessary) and refilling the reservoir and re-installing the spray nozzle. Recycling of the bomb spray would likely keep costs down and would encourage increased usage of the product.

Variations in the effects of the present invention can occur owing to the air flows created by heating and cooling system and by any impediments to the broadcast of the spray in the room such as furniture or stub walls or the like. Nonetheless the effects may be impacted but the overall treatment still is far more efficacious than reliance on the conventional method for preparing the examining room. It should be noted that the present invention is not necessarily intended to be used in lieu of actual wiping of primary surfaces. It is preferably supplemental to such activities. The minimal effort entailed in the use of the present invention will act as an incentive for its consistent use by the staff at a hospital or clinic. This is especially when time is at a premium during peak hours and the need to maintain sanitary conditions is at its highest.

The teachings of the present invention are not meant to be limiting but rather are meant to be illustrative of the potential ways in which the bomb spray can be used for disinfecting targeted areas. While the most obvious uses for the bomb spray of the present invention are examining and treatment rooms in hospital and clinical settings these are by no means the only applications that would benefit from the product. To that end, minor variations from the concept as taught herein are still intended to be within the scope of the invention.

I claim:

1. A bomb spray containing a propellant and a disinfectant chemical, where the bomb spray is capable of being activated in a room with surfaces causing the disinfectant chemical to be discharged, the bomb spray comprising:
A reservoir portion for containing the disinfectant chemistry where the volume of the reservoir can contain a sufficient amount of disinfectant chemistry to effectively contact the surfaces within the room when disinfectant chemistry is discharged;
A spray nozzle fitted onto the reservoir for fluid communication between the spray nozzle and the reservoir, where the spray nozzle further includes a control for operating the bomb spray between a stored condition and an activated condition, where in the activated condition the disinfectant chemistry is discharged through the spray nozzle; and,
Where the spray nozzle is optimized for the radial and vertical distribution of the disinfectant chemistry and is capable of broadcasting the disinfectant chemistry onto the surfaces within the room.

2. The bomb spray of claim 1, where the disinfectant chemistry is comprised of a formulation that includes at least a selection from the group of quarternary ammonium compounds.

3. The bomb spray of claim 1, where the spray nozzle is capable of broadcasting a portion of the spray laterally in a substantially 360 degree radial pattern while concurrently projecting a portion of the spray vertically.

4. The bomb spray of claim 1, where the reservoir is sized to contain sufficient disinfectant chemistry for distribution within a room used for medical services.

5. The bomb spray of claim 1, where the bomb spray may be re-used.

6. A bomb spray containing a propellant and a disinfectant chemical, where the bomb spray is capable of being activated in a room of the type and size used for treatment or examination of patients in a hospital or clinic and where the room includes both primary and secondary surfaces, causing the disinfectant chemical to be discharged, the bomb spray comprising:
A reservoir portion for containing the disinfectant chemistry where the volume of the reservoir can contain a sufficient amount of disinfectant chemistry to effectively contact the primary and secondary surfaces within the room when disinfectant chemistry is discharged;
A spray nozzle fitted onto the reservoir for fluid communication between the spray nozzle and the reservoir, where the spray nozzle further includes a control for operating the bomb spray between a stored condition and an activated condition, where in the activated condition the disinfectant chemistry is discharged through the spray nozzle; and,
Where the disinfectant chemistry is comprised of a formulation that includes at least a selection from the group of quarternary ammonium compounds and where the spray nozzle is optimized for the radial and vertical distribution of the disinfectant chemistry and is capable of broadcasting the disinfectant chemistry onto both the primary and secondary surfaces within the room.

7. A bomb spray of claim 6, where the quarternary ammonium compound is selected from the group of alkyl dimethyl benzyl ammonium chlorides.

8. The bomb spray of claim 7 where the particular alkyl dimethyl benzyyl ammonium chloride for use in the disinfectant formulation is preferably an alkyl dimethylbenzylammonium saccharinate.

9. The bomb spray of claim 6, where the disinfectant chemistry includes ethanol for the promotion of quick drying in the amount of approximately 75% of the total disinfectant chemistry formulation.

10. The bomb spray of claim 6, where the spray nozzle is capable of broadcasting a portion of the spray laterally in a substantially 360 degree radial pattern while concurrently projecting a portion of the spray vertically.

11. The bomb spray of claim 6, where the reservoir is sized for a single use.

12. The bomb spray of claim 11, where the reservoir is sized to contain sufficient disinfectant chemistry for distribution within a room used for medical services.

13. The bomb spray of claim 6, where the bomb spray may be re-used.

14. The bomb spray of claim 8 where the disinfectant chemistry is comprised of alkyl dimethylbenzylammonium saccharinate in the amount of approximately 1%, and ethanol in the amount of approximately 75%.

15. A method for the quick disinfection of the area within a room, where the room is of the type used for treatment or examination in a hospital or a clinic and where the room includes both primary and secondary surfaces, using a bomb spray that includes a disinfectant chemistry and a propellant, the steps of the method comprising
Preparing the room after it has been used, by removing used items or soiled items;
Acquiring a bomb spray of sufficient capacity to disperse the disinfectant onto substantially all of the primary and secondary surfaces in the room;
Placing the bomb spray substantially in the center of the room and at a height that is appropriate for allowing the spray to reach the primary and secondary surfaces;
Activating the bomb spray;
Allowing the bomb spray to completely discharge and thereafter allowing time for the disinfectant chemistry to contact the primary and secondary surfaces in the room;
Allowing time for the disinfectant chemistry to dry; and,
Opening the room again for service.

16. The method for quick disinfection of claim 15, where the disinfectant formulation is comprised of a quarternary ammonium compound that is selected from the group of alkyl dimethyl benzyl ammonium chlorides.

17. The method for quick disinfection of claim 16 where the particular alkyl dimethyl benzyl ammonium chloride for use in the disinfectant formulation is preferably an alkyl dimethylbenzylammonium saccharinate.

18. The method for quick disinfection of claim 15, where the bomb spray is sized for a single use.

19. The method for quick disinfection of claim 15, where the bomb spray may be re-used.
